(19) United States
(12) Patent Application Publication
(10) Pub. No.: US 2025/0345042 A1
(43) Pub. Date: Nov. 13, 2025

(54) PATHOGEN SAMPLE COLLECTION DEVICE

(71) Applicant: 3M INNOVATIVE PROPERTIES COMPANY, St. Paul, MN (US)

(72) Inventors: Audrey A. Sherman, Woodbury, MN (US); Narina Y. Stepanova, Woodbury, MN (US); Brett J. Sitter, Marine on St Croix, MN (US)

(21) Appl. No.: 18/868,435

(22) PCT Filed: May 9, 2023
(86) PCT No.: PCT/IB2023/054795

§ 371 Date: Nov. 22, 2024

Related U.S. Application Data
(60) Provisional application No. 63/346,375, filed on May 27, 2022.

(51) Int. Cl. A61B 10/00

(52) U.S. Cl. CPC A61B 10/00; A61B 2010/0087

(57) ABSTRACT

A pathogen sample collection device includes a liquid container defining a container volume and having an opening, and a volume of liquid eluent within the container volume and defining a liquid surface. An airflow conduit extends from an exhalation inlet end to a distal end. The airflow conduit extends through the opening. The distal end of the airflow conduit is disposed within the volume of liquid eluent or above the liquid surface, and the opening comprises an air outlet.

### FIELD

The present disclosure relates to a pathogen sample collection device. The present disclosure relates to a bioaerosol collection device.

### BACKGROUND

Diagnostic tests used to test for the presence of a virus or other pathogen in the airways, throat, or nasopharynx typically involve the insertion of a swab into the back of the nasal passage, the mid-turbinate area of the nasal passage, the anterior nares, or the throat to obtain a sample. The swab is then inserted into a container and analyzed or sent to a lab for processing. Other diagnostic tests involve collecting a saliva sample and then placing it in a container. Currently available at-home viral tests (e.g., COVID-19 tests) involve a nasal swab and a test kit (for example, the Ellume™ test, the Abbot™ BinaxNOW™ test, and the Lucira™ All-in-One test kit). Tests that utilize nasal swab samples or saliva contend with contaminants that can interfere with the various diagnostic tests. As a result, these sample types require a purification step when using RT-PCR molecular testing.

### SUMMARY

There is a need for an inexpensive, simple to use, and reliable pathogen sample collection system that may be used by laypeople to obtain a sample for testing for the presence of a target virus, target pathogen, or other target analyte, in a collected sample. The sample collection system may include a sample collection device for collecting a sample directly from exhalation airflow onto a sample collection liquid, which may be used to analyze the sample or to transfer the sample to a facility for testing.

It is desirable to provide a pathogen sample collection device that is easy to use. The device may advantageously be self-contained and optionally sterile. A self-contained (and optionally sterile) device may improve accuracy and reliability of pathogen testing due to the reduced contamination and background noise, unlike swabs and other test collection devices which may be contaminated upon use and/or during testing.

It is desirable to directly capture air-borne pathogens or biological samples without the need for a dry filter media that is then rinsed to collect the sample for testing. This wet media sample, after direct sample collection, may be directly tested or sealed and optionally transferred for testing safely without contamination.

The pathogen sample collection device enables direct capture of pathogens or biological samples from aerosols found in an exhalation breath, for example, into a wet or liquid media that may be directly tested or sealed for subsequent testing. By allowing the bioaerosol to directly impact onto the elution fluid, the sample can be captured therein without the need to capture the biological samples in any dry filtering device or dry swab that later must be rinsed of the collected sample. The collected sample may then be tested for a pathogen. Pathogen refers to an infectious microorganism or agent, such as a virus, bacterium, protozoan, prion, viroid, or fungus. In one exemplary embodiment the pathogen if a virus.

According to an embodiment, a pathogen sample collection device includes a liquid container defining a container volume and having an opening, and a volume of liquid eluent within the container volume and defining a liquid surface. An airflow conduit extends from an exhalation inlet end to a distal end. The airflow conduit extends through the opening. The distal end of the airflow conduit is disposed within the volume of liquid eluent or above the liquid surface, and the opening comprises an air outlet.

According to another embodiment, a pathogen sample collection device includes a liquid container defining a container volume and having an opening, and a volume of liquid eluent within the container volume and defining a liquid surface. An airflow conduit extends from an exhalation inlet end to a distal end. The airflow conduit is configured to allow only one way flow out of the distal end of the airflow conduit. The airflow conduit extends through the opening. The distal end of the airflow conduit is disposed within the volume of liquid eluent or above the liquid surface, and the opening comprises an air outlet.

According to another embodiment, a pathogen sample collection device includes a liquid container defining a container volume and having an opening. The container volume is less than 20 ml. A volume of liquid eluent within the container volume and defining a liquid surface. An airflow conduit extends from an exhalation inlet end to a distal end. The airflow conduit extends through the opening. The distal end of the airflow conduit is disposed within the volume of liquid eluent or above the liquid surface, and the opening comprises an air outlet.

The pathogen sample collection device is configured so that a user breathing into the airflow conduit forms a jet of exhalation airflow that contacts the volume of liquid eluent to capture a sample from the jet of exhalation airflow within the volume of liquid eluent. The distal end of the airflow conduit may be disposed within the volume of liquid eluent. The distal end of the airflow conduit may be disposed above the volume of liquid eluent or liquid surface. The container volume may be less than about 15 milliliters (ml) or in a range from about 1 to about 15 ml. The volume of liquid eluent may be less than 5 ml, or in a range from 1 ml to 5 ml.

According to another embodiment, a method of obtaining a pathogen sample using the sample collection device. The sample collection device includes a liquid container defining a container volume and having an opening, a volume of liquid eluent within the container volume and defining a liquid surface, and an airflow conduit extending from an exhalation inlet end to a distal end. The distal end of the airflow conduit is disposed within the volume of liquid eluent or above the liquid surface. The method includes breathing into the exhalation inlet end to form a jet of exhalation airflow out of the distal end of the airflow conduit. The jet of exhalation airflow contacting the volume of liquid eluent to capture a sample from the jet of exhalation airflow within the volume of liquid eluent and forming a liquid sample.

According to an embodiment, a kit for collecting a sample from breath includes the sample collection device and instructions for collecting a breath sample.

### BRIEF DESCRIPTION OF FIGURES

FIG. 1 is a schematic diagram side view of a pathogen sample collection device according to an embodiment.

FIG. 2 is a schematic diagram side view of a pathogen sample collection device collecting a pathogen sample.

FIG. 3 is a schematic diagram side view of a pathogen sample collection device where the airflow conduit is removed from the liquid container.

FIG. 4A is a schematic diagram side view of a pathogen sample collection device where the opening of the liquid container is sealed with a vial cap.

FIG. 4B is a schematic diagram side view of a pathogen sample collection device where the opening of the liquid container is closed with a dropper cap.

FIG. 5 is a schematic diagram side view of an embodiment of the airflow conduit including an air-porous layer disposed about the airflow conduit.

FIG. 6 is a schematic diagram side view of an embodiment of the airflow conduit including an angled coupling surface with radial ribs.

### DEFINITIONS

All scientific and technical terms used herein have meanings commonly used in the art unless otherwise specified. The definitions provided herein are to facilitate understanding of certain terms used frequently herein and are not meant to limit the scope of the present disclosure.

Unless otherwise indicated, the terms “polymer” and “polymeric material” include, but are not limited to, organic homopolymers, copolymers, such as for example, block, graft, random and alternating copolymers, terpolymers, etc., and blends and modifications thereof. Furthermore, unless otherwise specifically limited, the term “polymer” shall include all possible geometrical configurations of the material. These configurations include, but are not limited to, isotactic, syndiotactic, and atactic symmetries.

The terms “downstream” and “upstream” refer to a relative position based on a direction of exhalation airflow through the device. For example, the upstream-most element of the device is the exhalation inlet end, and the downstream-most element of the device is the outlet portion.

All headings provided herein are for the convenience of the reader and should not be used to limit the meaning of any text that follows the heading, unless so specified.

The term “i.e.” is used here as an abbreviation for the Latin phrase id est, and means “that is,” while “e.g.” is used as an abbreviation for the Latin phrase exempli gratia and means “for example.”

All scientific and technical terms used herein have meanings commonly used in the art unless otherwise specified. The definitions provided herein are to facilitate understanding of certain terms used frequently herein and are not meant to limit the scope of the present disclosure.

The term “about” is used here in conjunction with numeric values to include normal variations in measurements as expected by persons skilled in the art and is understood have the same meaning as “approximately” and to cover a typical margin of error, such as ±5% of the stated value. Moreover, unless otherwise indicated, all numbers expressing quantities, and all terms expressing direction/orientation (e.g., vertical, horizontal, parallel, perpendicular, etc.) in the specification and claims are to be understood as being modified in all instances by the term “about.”

Terms such as “a.” “an,” and “the” are not intended to refer to only a singular entity but include the general class of which a specific example may be used for illustration.

The terms “a,” “an,” and “the” are used interchangeably with the term “at least one.” The phrases “at least one of” and “comprises at least one of” followed by a list refers to any one of the items in the list and any combination of two or more items in the list.

As used here, the term “or” is generally employed in its usual sense including “and/or” unless the content clearly dictates otherwise. The term “and/or” means one or all of the listed elements or a combination of any two or more of the listed elements.

The recitations of numerical ranges by endpoints include all numbers subsumed within that range (e.g., 1 to 5 includes 1, 1.5, 2, 2.75, 3, 3.80, 4, 5, etc. or 10 or less includes 10, 9.4, 7.6, 5, 4.3, 2.9, 1.62, 0.3, etc.). Where a range of values is “up to” or “at least” a particular value, that value is included within the range.

As used here, “have”, “having”, “include”, “including”, “comprise”, “comprising” or the like are used in their open-ended sense, and generally mean “including, but not limited to.” It will be understood that “consisting essentially of,” “consisting of,” and the like are subsumed in “comprising” and the like. As used herein, “consisting essentially of,” as it relates to a composition, product, method or the like, means that the components of the composition, product, method or the like are limited to the enumerated components and any other components that do not materially affect the basic and novel characteristic(s) of the composition, product, method or the like.

The term “substantially” as used here has the same meaning as “significantly,” and can be understood to modify the term that follows by at least about 90%, at least about 95%, or at least about 98%. The term “not substantially” as used here has the same meaning as “not significantly,” and can be understood to have the inverse meaning of “substantially,” i.e., modifying the term that follows by not more than 10%, not more than 5%, or not more than 2%.

The words “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure, including the claims.

Any direction referred to here, such as “front,” “back,” “top,” “bottom,” “left,” “right,” “upper,” “lower,” and other directions and orientations are described herein for clarity in reference to the figures and are not to be limiting of an actual device or system or use of the device or system. Devices or systems as described herein may be used in a number of directions and orientations.

Any direction referred to here, such as “top,” “bottom,” “left,” “right,” “upper,” “lower,” and other directions and orientations are described herein for clarity in reference to the figures and are not to be limiting of an actual device or system or use of the device or system. Devices or systems as described herein may be used in a number of directions and orientations.

The term “pathogen” refers to an infectious microorganism or agent, such as a virus, bacterium, protozoan, prion, viroid, or fungus.

### DETAILED DESCRIPTION

The present disclosure relates to a pathogen sample collection device. The present disclosure relates to a bioaerosol sample collection device.

The pathogen sample collection device described herein directly captures air-borne pathogens or biological samples with a wet media or eluent without the need for a dry filter media. This wet media sample, after direct sample collection, may be directly tested or sealed and optionally transferred for testing safely without contamination. Directly capturing the air-borne pathogens or biological samples with a wet media or eluent that can be directly tested reduces or eliminate contamination potential and error and improves reliability of the sample and test.

The pathogen sample collection device described herein eliminates the need for any dry media currently used to capture pathogens or biological samples from a consumer's airway or exhalation breath.

The pathogen sample collection device includes a liquid container defining a container volume and having an opening, a volume of liquid eluent within the container volume and defining a liquid surface, and an airflow conduit extending from an exhalation inlet end to a distal end. The distal end of the airflow conduit is disposed within the volume of liquid eluent or above the liquid surface.

The pathogen sample collection device includes a liquid container defining a container volume and having an opening, a volume of liquid eluent within the container volume and defining a liquid surface. and an airflow conduit extending from an exhalation inlet end to a distal end. The airflow conduit may be configured to allow only one-way flow out of the distal end. The distal end of the airflow conduit is disposed within the volume of liquid eluent or above the liquid surface.

The pathogen sample collection device includes a liquid container defining a container volume and having an opening. The container volume may be less than 20 ml. A volume of liquid eluent within the container volume and defining a liquid surface. An airflow conduit extends from an exhalation inlet end to a distal end. The airflow conduit extends through the opening. The distal end of the airflow conduit is disposed within the volume of liquid eluent or above the liquid surface, and the opening comprises an air outlet. The distal end of the airflow conduit may be co-planar with the liquid surface.

The airflow conduit has an exhalation inlet end that may be configured to mate with the consumer's mouth, or nose, as desired. The distal end of the airflow conduit may be adjacent to the liquid surface or liquid level of the volume of liquid eluent. The distal end of the airflow conduit may be separated from the liquid surface. The distal end of the airflow conduit may be separated from the liquid surface by a distance of at least 1 ml, or in a range from 1 ml to 10 ml, or in a range from 1 ml to 5ml, or in a range from 1 ml to 3 ml.

The distal end of the airflow conduit may be disposed within the liquid surface or liquid level of the volume of liquid eluent. The distal end of the airflow conduit may be above the liquid surface or liquid level of the volume of liquid eluent. Applicant has found that a jet of exhalation air impinging into the surface of the volume of liquid eluent captures pathogens or biological samples from an exhalation breath of a consumer.

The airflow conduit may have a first open diameter at the exhalation inlet end and a second open diameter at the distal end, and the second open diameter is less than the first open diameter. This configuration may increase a velocity of the exhalation air flowing out of the distal end of the airflow conduit as compared to the airflow velocity into the exhalation inlet end. This increased velocity of exhalation air flowing out of the distal end of the airflow conduit may form a jet of exhalation airflow that may impinge onto or into the liquid surface of the volume of liquid eluent.

The airflow conduit may include a lid or coupling feature to mate with the opening of the liquid container. The coupling feature and the opening of the liquid container may have complimentary coupling features to provide a secure attachment of the coupling feature with the opening of the liquid container. The airflow conduit may be integrally formed with the lid or coupling feature. The airflow conduit may be fixed to the lid or coupling feature.

The lid or coupling feature may be configured to engage with the opening with a snap-fit feature. The lid or coupling feature may be configured to engage with the opening with a threaded coupling mechanism or a screw fit feature. Both the opening and the lid or coupling feature may have complimentary threaded coupling mechanisms or screw fit features.

This lid or coupling feature may include an air outlet to allow air to escape from the liquid container once the airflow conduit is mated to the liquid container. The terms “lid” and “coupling feature” are used interchangeable herein.

The coupling feature may have an angled surface, such as a frustoconical shape that mates with the opening of the liquid container. The coupling feature may have one or more radial ribs that engage with the opening of the liquid container. The radial ribs may include a first rib adjacent the coupling end and having a first diameter, and a second rib having a second diameter that is greater than the first diameter.

The airflow conduit may be configured to allow only one-way flow out of the distal end. This feature mitigates or prevents back flow of liquid eluent towards the exhalation inlet end of the airflow conduit. The airflow conduit may include a one-way flow valve, or a check valve. The one-way flow valve may include a ball-type check valve, a swing-type check valve, an in-line check valve, a tilting disc check valve, a lift-type check valve, and the like.

The exhalation inlet end of the airflow conduit may form an exhalation piece or couple to an exhalation piece. such as a mouthpiece or a nosepiece. The exhalation piece may be configured to accommodate exhalation through either the mouth or the nose. A nasal exhalation piece may be configured to enclose a consumer's nose and allow nasal exhalation to be collected and transmitted through the airflow conduit of the pathogen sample collection device.

The airflow conduit coupling feature may mate or couple with the opening of the liquid container with any suitable mechanism. For example, the coupling feature and opening may be coupled by bayonet coupling, interference fit, snap fit, or threaded coupling. In one embodiment, the coupling feature and opening are coupled by bayonet coupling. When configured for bayonet coupling, the coupling feature may include one or more protrusions and the opening may include one or more corresponding grooves constructed to receive and guide the one or more protrusions. Alternatively, the one or more protrusions may be on the opening and the one or more grooves may be on the coupling feature.

The airflow conduit and liquid container may be formed of any suitable material. The housing may be formed of a rigid material, such as plastic, metal, glass, or the like, or a combination thereof.

The liquid container may contain a small volume of liquid. The liquid container may have a volume that is typical of medical collection vials. The liquid container may have a volume in a of less than 50 ml, or less than 40 ml, or less than 30 ml, or less than 20 ml, or less than 15 ml, or in a range from 1 ml to 50 ml, or in a range from 2 ml to 20 ml, or in a range from 5 ml to 10 ml.

The volume of liquid eluent may be less than 25 ml, or less than 15 ml, or less than 10 ml, or less than 5 ml, or in a range from 1 ml to 25 ml, or in a range from 1 ml to 10 ml, or in a range from 1 ml to 5 ml. Collection of a pathogen sample may be more effective in a small volume of liquid eluent, as the sample is concentrated in a small volume.

The sample collection system may be provided as a kit. The kit may include the sample collection device as discussed above and one or more opening adapters configured to either seal the collected sample or direct the sample into a testing device. For example, once the sample is collected and the airflow conduit is removed from the liquid container, the consumer may couple a seal lid into the opening of the liquid container to preserve the collected sample for subsequent testing. Alternatively or in addition, once the sample is collected and the airflow conduit is removed from the liquid container, the consumer may couple a dropper lid into the opening of the liquid container to dropwise dispense the collected sample onto a testing device.

The liquid eluent may be an aqueous liquid. The liquid eluent may be a buffer solution. The liquid eluent may be an aqueous buffer solution. The liquid eluent may be a saline solution. The liquid eluent may include a surfactant. The liquid eluent (e.g., a buffer or a saline solution) may include from 0.1 wt-% or more or 0.5 wt-% or more, and up to 1 wt-% or up to 2 wt-% of surfactant.

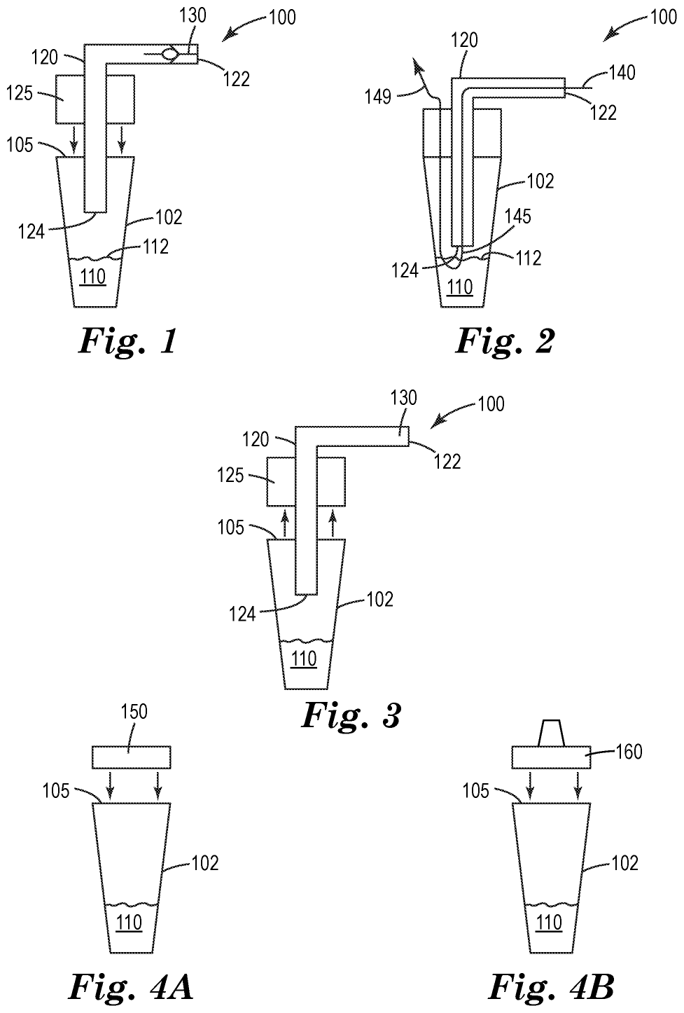

FIG. 1 is a schematic diagram side view of a pathogen sample collection device 100 according to an embodiment. FIG. 2 is a schematic diagram side view of a pathogen sample collection device 100 collecting a pathogen sample. FIG. 3 is a schematic diagram side view of a pathogen sample collection device 100 where the airflow conduit 120 is removed from the liquid container 102.

The pathogen sample collection device includes a liquid container 102 defining a container volume and having an opening 105. The container volume may be less than 20 ml. A volume of liquid eluent 110 is held within the container volume and defines a liquid surface 112. An airflow conduit 120 extends from an exhalation inlet end 122 to a distal end 124. The airflow conduit 120 may be configured to allow only one-way flow out of the distal end 124. The distal end 124 of the airflow conduit is disposed within the volume of liquid eluent 110 or above the liquid surface 112.

The airflow conduit 120 is configured to allow only one-way flow out of the distal end 124. This feature mitigates or prevents back flow of liquid eluent 110 towards the exhalation inlet end 122 of the airflow conduit 120. The airflow conduit 120 may include a one-way flow valve 130, or a check valve 130. The one-way flow valve 130 may include a ball-type check valve, a swing-type check valve, an in-line check valve, a tilting disc check valve, a lift-type check valve, and the like.

The distal end 124 of the airflow conduit 120 may be disposed within the volume of liquid eluent 110 where the entire distal end 124 of the airflow conduit 120 is submerged within the volume of liquid eluent 110. In these embodiments, exhalation airflow 140 enters the exhalation inlet end 122 and flows through the airflow conduit 120 to a distal end 124. This exhalation airflow exiting the distal end 124 forms a jet of exhalation airflow 145 that penetrates the volume of liquid eluent 110. Pathogen or biological sample is thus captured by the liquid eluent 110. Exhalation airflow moved from the liquid eluent 110 and out of the liquid container 102 forming exhalation outlet airflow 149 via an optional air outlet through the coupling feature 125.

The distal end 124 of the airflow conduit 120 may be co-planar with the liquid surface 112 of the liquid eluent 110. where the distal end 124 of the airflow conduit 120 is not below the liquid surface 112 of the liquid eluent 110. In these embodiments, exhalation airflow 140 enters the exhalation inlet end 122 and flows through the airflow conduit 120 to a distal end 124. This exhalation airflow exiting the distal end 124 forms a jet of exhalation airflow 145 that impinges onto the surface and may penetrate the liquid surface 112 of the liquid eluent 110. Pathogen or biological sample is thus captured by the liquid eluent 110. Exhalation airflow moved from the liquid eluent 110 and out of the liquid container 102 forming exhalation outlet airflow 149 via an optional air outlet through the coupling feature 125.

The distal end 124 of the airflow conduit 120 may be above the liquid surface 112 of the liquid eluent 110, so that the distal end 124 is spaced away from the liquid surface 112 of the liquid eluent 110. In these embodiments, exhalation airflow 140 enters the exhalation inlet end 122 and flows through the airflow conduit 120 to a distal end 124. This exhalation airflow exiting the distal end 124 forms a jet of exhalation airflow 145 that impinges onto the surface and may penetrate the liquid surface 112 of the liquid eluent 110. Pathogen or biological sample is thus captured by the liquid eluent 110. Exhalation airflow moved from the liquid eluent 110 and out of the liquid container 102 forming exhalation outlet airflow 149 via an optional air outlet through the coupling feature 125.

Once the pathogen sample is collected directly into the volume of liquid eluent 110, the airflow conduit 120 is removed from the liquid container 102 and then tested or sealed and subsequently tested.

FIG. 4A is a schematic diagram side view of a pathogen sample collection device 100 where the opening 105 of the liquid container 102 is sealed with a vial or seal cap 150. FIG. 4B is a schematic diagram side view of a pathogen sample collection device 100 where the opening 105 of the liquid container 102 is closed with a dropper cap 160.

The seal cap 150 or dropper cap 160 may be coupled to the opening 105 via any useful mechanism. For example, the seal cap 150 or dropper cap 160 may be coupled to the opening 105 by bayonet coupling. interference fit, snap fit, or threaded coupling.

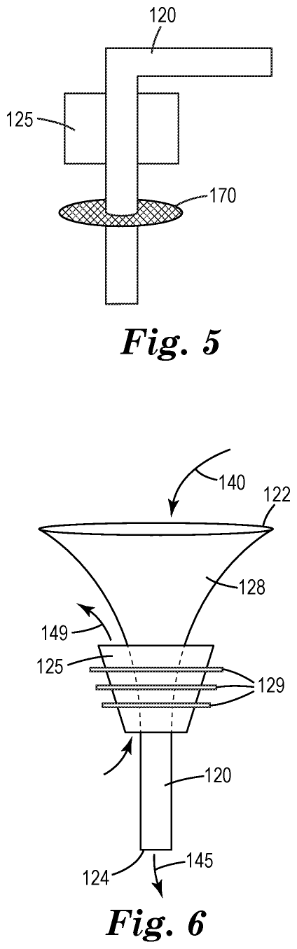

FIG. 5 is a schematic diagram side view of an embodiment of the airflow conduit 120 including an optional air-porous layer 170 disposed about the airflow conduit 120. When present, the air-porous layer 170 may function as a liquid barrier to prevent bubbles or liquid spattering from exiting the liquid container 102 during pathogen sample collection. The air-porous layer 170 may not absorb liquid eluent 110. The air-porous layer 170 may be disposed between the liquid surface 112 and the opening 105 of the liquid container 102. The air-porous layer 170 may substantially extend along an entire inner diameter of the liquid container 102.

FIG. 6 is a schematic diagram side view of an embodiment of the airflow conduit 120 including a nasal feature 128 and an angled coupling surface 125 with ribs 129. The coupling mechanism 125 may define a frustoconical portion and a plurality of ribs 129 extending from the frustoconical portion. The plurality of ribs 129 may include radial ribs 129. The radial ribs 129 may define a first rib adjacent the coupling end and having a first diameter, and a second rib having a second diameter that is greater than the first diameter, as illustrated in FIG. 6.

In the embodiment shown, the coupling feature 125 includes a frustoconical portion and one or more rings or ribs 129 extending outwardly from the frustoconical portion. The frustoconical portion may be tapered toward one end, as shown. The ribs 129 engage at least partially with the inside surface of the opening 105 of the liquid container 102. According to an embodiment, the ribs 129 may be deformable. That is, when the coupling feature 125 is inserted into the opening 105 of the liquid container 102. the ribs 129 are deformed past their yield point to provide the interference fit to the opening 105 of the liquid container 102. The frustoconical portion 125 may also be formed by a thin wall that provides additional flexibility to help with the fitment.

The plurality of ribs 129 may have different sizes or diameters. The size of the ribs 129 may gradually increase. beginning with the smallest diameter adjacent one end and ending with the largest diameter adjacent the opposing end. That is, the plurality of ribs 29 may include at least a first rib adjacent the one end having a first diameter, and a second rib further away from the one end and having a second diameter that is greater than the first diameter. The plurality of ribs 129 may further include additional ribs with progressively increasing diameters. The different sizes of the ribs 129 and the tapered frustoconical portion 125 allow connection to the opening 105 of the liquid container 102 of different inside diameters without concern about the specific dimensions of threads or outside diameter of the coupling feature 125.

In the embodiment shown, the ribs 129 are radial ribs that extend circumferentially around the frustoconical portion 125. However, in some embodiments the ribs 129 have a different shape that facilitates a mechanical interference fit with the inside of a liquid container 102. For example, the ribs 129 may extend only partially around the frustoconical portion 125, or may form fins extending axially along the frustoconical portion 125, or may form a wedge.

The pathogen sample collection device 100 may be utilized by a consumer by the consumer exhaling, either though the nose or mouth into the exhalation inlet end 122 of the airflow conduit 120 and impinging this exhalation airflow 145 or jet of exhalation airflow 145 out of the distal end 124 of the airflow conduit 120. The jet of exhalation airflow 145 contacts the volume of liquid eluent 110 to capture a sample from the jet of exhalation airflow 145 within the volume of liquid eluent 110.

The consumer may then test the collected sample or seal the collected sample for subsequent testing.

The sample collection device may further comprise a machine-readable optical label. Such labels may include, for example, a bar code and a QR (quick response) code. The machine-readable optical label may be configured to identify the device. An electronic reader capable of reading machine-readable optical labels may be used to read and record the result. An electronic reader may be, for example, a smart phone, a tablet, a laptop, or bar code reader or QR code reader. The electronic reader may further be used to transmit the result, for example, to a healthcare provider or to a database.

In another configuration, a kit may include the sample collection device and instructions for collecting a sample. The instructions may include instructions to: exhale into the sample collection device and then test the liquid sample or seal the liquid sample for subsequent testing. The instructions may further include instructions to read an optical label using an electronic reader.

The instructions may include instructions for collecting a pathogen from the pathogen sample. The kit may include a pathogen testing device and instructions for testing the pathogen sample. The pathogen testing device may be a liquid assay device. The liquid assay device may be a vertical flow assay device, or a lateral flow assay device, for example. In one embodiment, the instructions include testing for a virus in the pathogen sample.

All references and publications cited herein are expressly incorporated herein by reference in their entirety into this disclosure, except to the extent they may directly contradict this disclosure. Although specific embodiments have been illustrated and described herein, it will be appreciated by those of ordinary skill in the art that a variety of alternate and/or equivalent implementations can be substituted for the specific embodiments shown and described without departing from the scope of the present disclosure. It should be understood that this disclosure is not intended to be unduly limited by the illustrative embodiments and examples set forth herein and that such examples and embodiments are presented by way of example only with the scope of the disclosure intended to be limited only by the claims set forth here.

1. A pathogen sample collection device comprising:
a liquid container defining a container volume and having an opening, the container volume being less than 20 ml;
a volume of liquid eluent within the container volume and defining a liquid surface; and
an airflow conduit extending from an exhalation inlet end to a distal end, the airflow conduit extending through the opening;
wherein the distal end of the airflow conduit is disposed within the volume of liquid eluent or above the liquid surface, and the opening comprises an air outlet.

2. A pathogen sample collection device comprising:
a liquid container defining a container volume and having an opening;
a volume of liquid eluent within the container volume and defining a liquid surface; and
an airflow conduit extending from an exhalation inlet end to a distal end, the airflow conduit configured to allow only one way flow out of the distal end of the airflow conduit, the airflow conduit extending through the opening;
wherein the distal end of the airflow conduit is disposed within the volume of liquid eluent or above the liquid surface, and the opening comprises an air outlet.

3. The pathogen sample collection device of claim 1, wherein the airflow conduit is configured to allow only one way flow out of the distal end of the airflow conduit.

4. The pathogen sample collection device of claim 1, wherein the container volume is less than 15 ml, or in a range from 5 ml to 10 ml.

5. The pathogen sample collection device of a claim 1, wherein the volume of liquid eluent is less than 5 ml, or in a range from 1 ml to 5 ml.

6. The pathogen sample collection device of claim 1, wherein the airflow conduit is disposed within the volume of liquid eluent.

7. The pathogen sample collection device of claim 1, wherein the distal end of the airflow conduit is above the liquid surface.

8. The pathogen sample collection device of claim 1, wherein the airflow conduit has a first open diameter at the exhalation inlet end and a second open diameter at the distal end, and the second open diameter is less than the first open diameter.

9. The pathogen sample collection device of claim 1, wherein the airflow conduit comprises a one-way flow valve.

10. The pathogen sample collection device of a claim 1, further comprising a lid configured to mate with the opening.

11. The pathogen sample collection device of claim 10, wherein the airflow conduit extends through the lid.

12. The pathogen sample collection device of claim 10, wherein the lid comprises an air outlet.

13. The pathogen sample collection device of claim 1, wherein the lid engages with the opening with a snap-fit.

14. The pathogen sample collection device of claim 1, wherein the lid comprises a coupling feature to mate with a complimentary coupling feature at the opening of the liquid container.

15. The pathogen sample collection device of claim 14, wherein the coupling feature comprises a threaded coupling mechanism and the complimentary coupling feature is a threaded coupling mechanism.

16. The pathogen sample collection device of claim 14, wherein the coupling feature comprises an angled coupling surface.

17. The pathogen sample collection device of claim 14, wherein the coupling feature comprises frustoconical portion and a plurality of ribs extending from the frustoconical portion.

18. The pathogen sample collection device of claim 17, wherein the plurality of ribs comprises radial ribs.

19. The pathogen sample collection device of claim 17, wherein the plurality of ribs comprise a first rib adjacent a first end and having a first diameter, and a second rib having a second diameter that is greater than the first diameter.

20. The pathogen sample collection device of any preceding claim, wherein the liquid eluent is a saline solution.

21-40. (canceled)
